(19) United States
(12) Patent Application Publication
(10) Pub. No.: US 2012/0003154 A1
(43) Pub. Date: Jan. 5, 2012

(54) ARYLOXYANILIDE DERIVATIVES

(76) Inventors: Harry John Wadsworth, Amersham (GB); William John Trigg, Amersham (GB)

(21) Appl. No.: 13/256,678

(22) PCT Filed: Mar. 19, 2010
(86) PCT No.: PCT/EP2010/053614

§ 371 Date: Sep. 15, 2011

Related U.S. Application Data
(60) Provisional application No. 61/161,425, filed on Mar. 19, 2009.

(30) Foreign Application Priority Data
Mar. 19, 2009 (GB) 0904715.0

(51) Int. Cl. A61K 51/04; B65D 77/00; B01J 19/00; C07D 213/75; C07D 213/64

(52) U.S. Cl. 424/1.89; 546/297; 546/300; 422/159; 206/216

(57) ABSTRACT

The present invention provides a novel radiolabeled aryloxyalinine derivative suitable for in vivo imaging. In comparison to known aryloxyalinine derivative in vivo imaging agents, the in vivo imaging agent of the present invention has better properties for in vivo imaging. The in vivo imaging agent of the present invention demonstrates good selective binding to the peripheral benzodiazepine receptor (PBR), in combination with good brain uptake and in vivo kinetics following administration to a subject.

### TECHNICAL FIELD OF THE INVENTION

The present invention concerns in vivo imaging and in particular in vivo imaging of peripheral benzodiazepine receptors (PBR). An aryloxyanilide in vivo imaging agent is provided that binds with nanomolar affinity to PBR, has good uptake into the brain following administration, and which has good selective binding to PBR. The present invention also provides a precursor compound useful in the synthesis of the in vivo imaging agent of the invention, as well as a method for synthesis of said in vivo imaging agent comprising use of said precursor compound, and a kit for carrying out said method. A cassette for the automated synthesis of the in vivo imaging agent is also provided. In addition, the invention provides a radiopharmaceutical composition comprising the in vivo imaging agent of the invention, as well as methods for the use of said in vivo imaging agent.

### DESCRIPTION OF RELATED ART

The peripheral benzodiazepine receptor (PBR) is known to be mainly localised in peripheral tissues and glial cells but its physiological function remains to be clearly elucidated. Subcellularly, PBR is known to localise on the outer mitochondrial membrane, indicating a potential role in the modulation of mitochondrial function and in the immune system. It has furthermore been postulated that PBR is involved in cell proliferation, steroidogenesis, calcium flow and cellular respiration. PBR has been associated with a variety of conditions including acute and chronic stress, anxiety, depression, Parkinson's disease, Alzheimer's disease, brain damage, cancer (Gavish et al Pharm. Rev. 1999; 51: 629), Huntington's disease (Meβmer and Reynolds Neurosci. Lett. 1998; 241: 53-6), asthma (Pelaia et al Gen. Pharmacol. 1997; 28(4): 495-8), rheumatoid arthritis (Bribes et al Eur. J. Pharmacol. 2002; 452(1): 111-22), atherosclerosis (Davies et al J. Nucl. Med. 2004; 45: 1898-1907) and multiple sclerosis (Banati et al 2000 Brain; 123: 2321). PBR may also be associated with neuropathic pain, Tsuda et al having observed activated microglia in subjects with neuropathic pain (2005 TINS 28(2) pp 101-7).

Positron emission tomography (PET) imaging using the PBR selective ligand, (R)-[¹¹C]PK11195 provides a generic indicator of central nervous system (CNS) inflammation. Despite the successful use of (R)-[¹¹C]PK11195, it has its limitations. It is known to have high protein binding, and low specific to non-specific binding. The role of its radiolabelled metabolites is not known and quantification of binding requires complex modelling. There have been efforts to provide compounds having high affinity and selectivity for PBR to enable improved measurement of PBR in the CNS.

Aryloxyalinine derivatives have been proposed that have high affinity for PBR, as well as high selectivity for PBR over the central benzodiazepine receptor (CBR) (Chaki et al 1999 Eur. J. Pharmacol.; 371: 197-204). [¹¹C]-DAA1106 and [¹⁸F]-FE-DAA1106 are PET radioligands based on these aryloxyalinine compounds. These PET radioligands are taught in U.S. Pat. No. 6,870,069, and have been studied in humans (Ikomo et al J. Cereb. Blood Flow Metab. 2007; 27: 173-84 and Fujimura et al J. Nuc. Med. 2006; 47: 43-50). Alternative radiofluorinated DAA1106 derivatives are taught in WO 2007/074383. Alternative ¹¹C-labelled DAA1106 derivatives are described in WO 2007/036785. Radioiodinated DAA1106 is described in EP 1854781, and by Zhang et al (2007 J. Med. Chem.; 50: 848-55). The chemical structures of [¹¹C]-DAA1106, [¹⁸F]-FE-DAA1106 and [¹²³I]-DAA1106 are as follows:

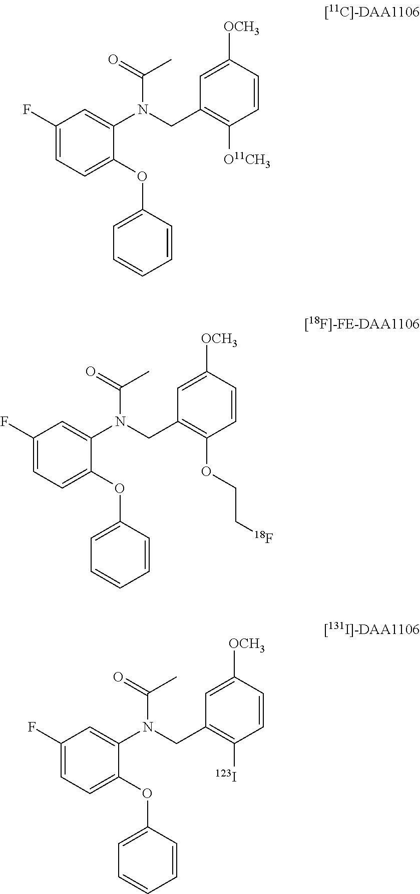

However, the kinetic properties of these compounds are not ideal for in vivo imaging such that their application to quantitative studies may be limited.

In an effort to improve further upon the DAA1106 series of radioligands, another aryloxyaniline derivative, PBR28, has been reported by Briard et al (J. Med. Chem. 2008; 51: 17-30). The structures of PBR28 and PET radioligands derived from PBR28 are as follows:

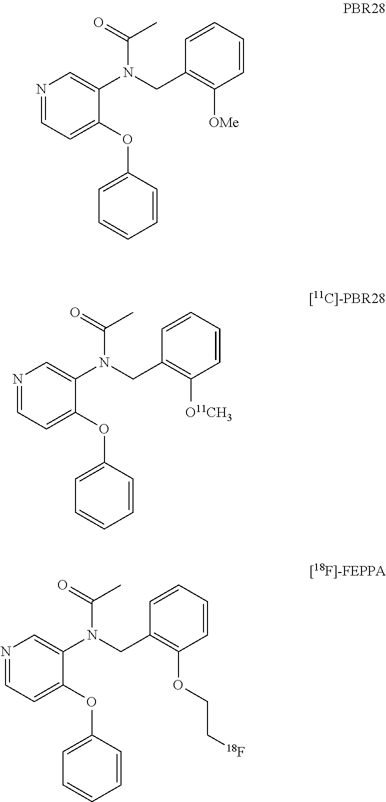

[¹⁸F]-FEPPA (also known as [¹⁸F]-FE-PBR28) was found to have subnanomolar affinity for PBR in vitro, and showed good uptake into the brain of naive rats following intravenous injection (Wilson et al Nuc. Med. Biol. 2008; 35: 305-14), although sensitivity and specificity were not determined. [¹¹C]-PBR28 has been studied in monkey to assess its brain kinetics using PET. [¹¹C]-PBR28 was reported by Briard et al (supra) to have high brain uptake, good specific binding to PBR-expressing tissues and kinetic properties more suitable for in vivo imaging as compared with (R)-[¹¹C]PK11195. The present inventors have found that although PBR28 demonstrates better properties as an in vivo PBR imaging agent compared with (R)-[¹¹C]-PK11195, its specificity for PBR-expressing tissues is not ideal. Furthermore, the present inventors have found that the in vivo clearance properties of PBR28 are also not ideal. There is therefore scope to provide a further improved PBR-specific in vivo imaging agent.

WO 2010/015340 and WO 2010/015387, published after the priority date of the present application, disclose a further class of aryloxyalinine derivatives having a nitrogen heteroatom on the same ring as PBR28, but at a different position in that ring. WO 2010/015340 and WO 2010/015387 broadly disclose a compound of formula I:

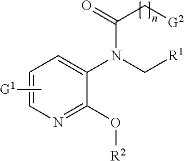

wherein:

R¹ and R² are independently and individually, at each occurrence, selected from the group consisting of (G³)aryl, substituted (G³)aryl, (G³-(C₁-C₈)alkyl)aryl, (G³-(C₁-C₈)alkoxy)aryl, (G³-(C₂-C₈)alkynyl)aryl, (G³-(C₂-C₈)alkenyl)aryl, substituted (G³-(C₁-C₈)alkyl)aryl, substituted (G³-(C₁-C₈)alkoxy)aryl, substituted (G³-(C₂-C₈)alkynyl)aryl and substituted (G³-(C₂-C₈)alkenyl)aryl,

G¹, G² and G³ are independently and individually, at each occurrence, selected from the group consisting of hydrogen and L, with the proviso that compounds of formula I contain exactly one L;

L is selected from the group consisting of R³, [¹⁸F]fluoro and [¹⁹F]fluoro;

R³ is a leaving group;

wherein n is an integer from 0 to 6.

Data is provided in WO 2010/015340 and WO 2010/015387 to show that two particular compounds (named “2d” and “5d”) have improved properties for in vivo imaging of PBR in the brain as compared with previous aryloxyalinine derivatives. The chemical formulae of these two compounds are provided below:

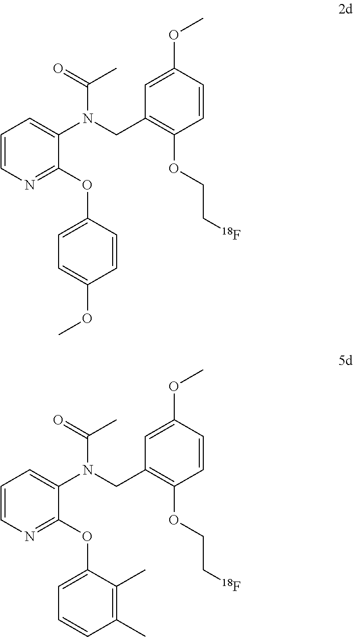

### SUMMARY OF THE INVENTION

The present invention provides a novel radiolabelled aryloxyalinine derivatives suitable for in vivo imaging. The in vivo imaging agents of the present invention have good properties for in vivo imaging the peripheral benzodiazepine receptor (PBR) in the central nervous system (CNS). The in vivo imaging agent of the present invention demonstrates good selective binding to PBR, in combination with good brain uptake and in vivo kinetics following administration to a subject.

### DETAILED DESCRIPTION OF THE INVENTION

Imaging Agent

In one aspect, the present invention provides an in vivo imaging agent of Formula I:

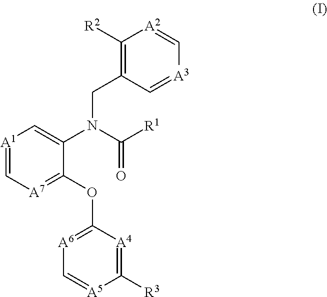

or a salt or solvate thereof, wherein:
R¹ is selected from methyl and C₁₋₃ fluoroalkyl;
R² is selected from hydrogen, halogen, C₁₋₃ alkoxy, and C₁₋₃ fluoroalkoxy;
R³ is selected from hydrogen, halogen and C₁₋₃ alkoxy;
A¹ is CH or N;
1 or 2 of A² and A⁴⁻⁷ are N, and the rest of A² and A⁴⁻⁷ are CH;
A³ is CH or CH—O—R⁴, where R⁴ is H, C₁₋₃ alkyl or C₁₋₃ haloalkyl, or A³ can alternatively be N when one of A² and A⁴⁻⁷ is N, and the rest of A² and A⁴⁻⁷ are CH;
and wherein Formula I as defined comprises an atom which is a radioisotope suitable for in vivo imaging.

An “in vivo imaging agent” in the context of the present invention refers to a radiolabelled compound suitable for in vivo imaging. The term “in vivo imaging” as used herein refers to those techniques that non-invasively produce images of all or part of the internal aspect of a subject. Examples of such in vivo imaging methods are single photon emission computed tomography (SPECT) and positron emission tomography (PET).

Suitable salts according to the invention include (i) physiologically acceptable acid addition salts such as those derived from mineral acids, for example hydrochloric, hydrobromic, phosphoric, metaphosphoric, nitric and sulphuric acids, and those derived from organic acids, for example tartaric, trifluoroacetic, citric, malic, lactic, fumaric, benzoic, glycollic, gluconic, succinic, methanesulphonic, and para-toluenesulphonic acids; and (ii) physiologically acceptable base salts such as ammonium salts, alkali metal salts (for example those of sodium and potassium), alkaline earth metal salts (for example those of calcium and magnesium), salts with organic bases such as triethanolamine, N-methyl-D-glucamine, piperidine, pyridine, piperazine, and morpholine, and salts with amino acids such as arginine and lysine.

Suitable solvates according to the invention include those formed with ethanol, water, saline, physiological buffer and glycol.

Unless otherwise specified, the term “alkyl” alone or in combination, means a straight-chain or branched-chain alkyl radical containing preferably from 1 to 3 carbon atoms. Examples of such radicals include, methyl, ethyl, and propyl.

Unless otherwise specified, the term “alkoxy”, alone or in combination, means an alkyl ether radical of formula —O-alkyl wherein the term alkyl is as defined above. Examples of suitable alkyl ether radicals include, methoxy, ethoxy, and propoxy.

The term “halogen” or “halo-” means a substituent selected from fluorine, chlorine, bromine or iodine. “Haloalkyl” and “haloalkoxy” are alkyl and alkoxy groups, respectively, as defined above substituted with one or more halogens, preferably at the terminal end, i.e. -alkyl-halogen and -alkoxy-halogen, respectively.

The term “comprises an atom which is a radioisotope suitable for in vivo imaging” means that in Formula I as defined above, the isotopic form of one of the atoms is a radioisotope suitable for in vivo imaging. In order to be suitable for in vivo imaging, the radioisotope is detectable externally following administration to said subject. Preferred radioisotopes of the present invention are gamma-emitting radioactive halogens and positron-emitting radioactive non-metals. Examples of gamma-emitting radioactive halogens suitable for use in the present invention are ¹²³I, ¹³¹I and ⁷⁷Br. A preferred gamma-emitting radioactive halogen is ¹²³I. Examples of positron-emitting radioactive non-metal suitable for use in the present invention are ¹¹C, ¹³N, ¹⁸F and ¹²⁴I. Preferred positron-emitting radioactive non-metals are ¹¹C and ¹⁸F, and in particular ¹⁸F.

Preferably, R¹ is C₁₋₃ fluoroalkyl and R² is hydrogen; or, R¹ is methyl and R² is C₁₋₃ fluoroalkoxy.

R³ is preferably hydrogen.

In one preferred embodiment of the in vivo imaging agent of Formula I, 2 of A¹, A² and A⁴⁻⁷ are N and the rest of A¹, A² and A⁴⁻⁷ are CH.

In another preferred embodiment of the in vivo imaging agent of Formula I, 1 of A² and A⁴-A⁶ is N; A¹ is CH; and, A⁷ is CH.

In a yet further preferred embodiment of the in vivo imaging agent of Formula I, A⁷ is N; A¹⁻⁶ are CH; and, R³ is hydrogen.

A preferred radioisotope suitable for in vivo imaging for the present invention is ¹⁸F. Most preferably, either R¹ is [¹⁸F]fluoroalkyl, or R² is [¹⁸F]fluoroalkoxy. Examples of such in vivo imaging agents are imaging agents 1-19 as follows:

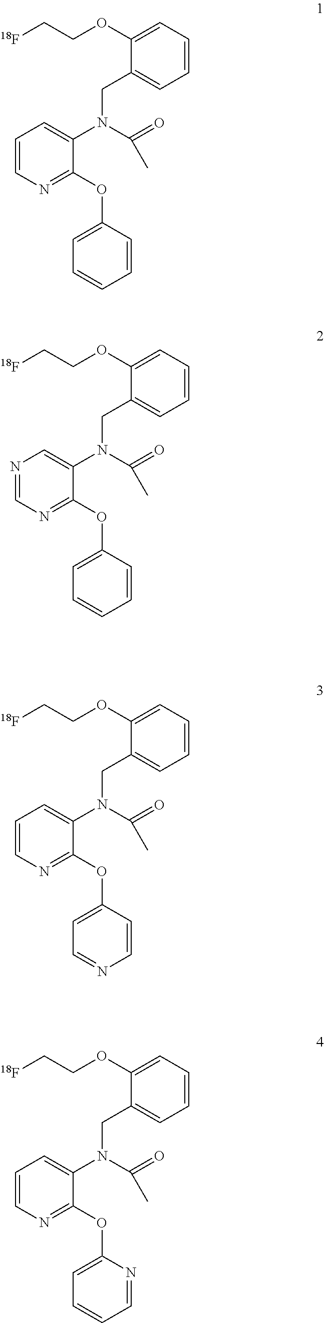

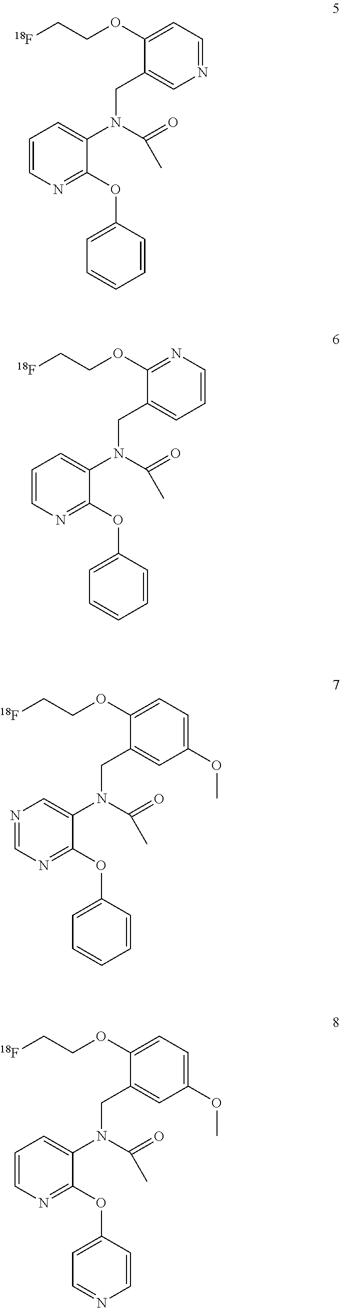

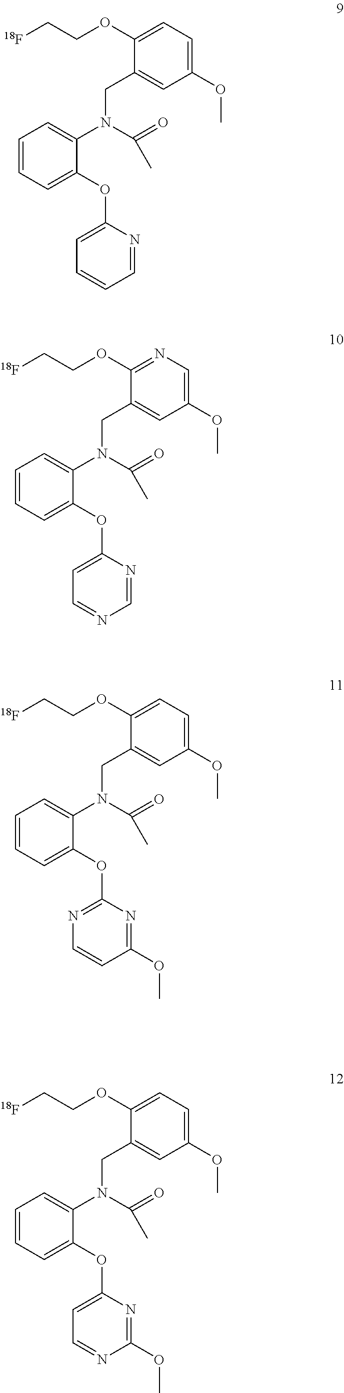

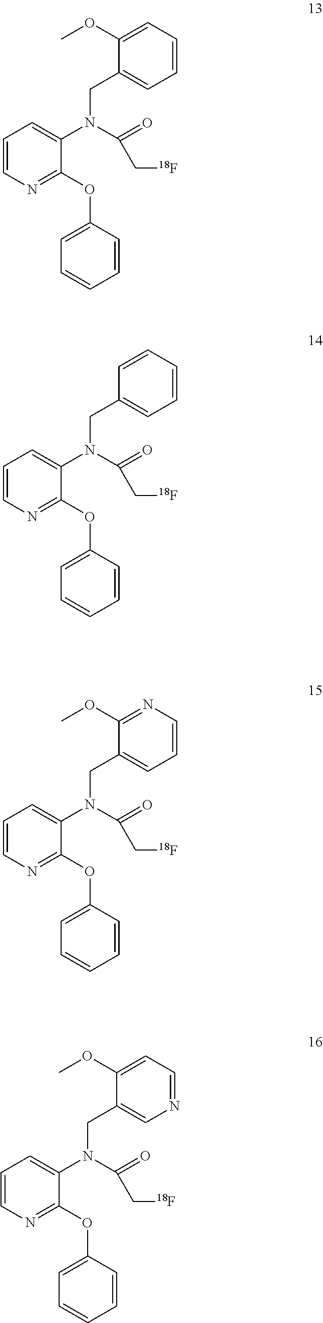

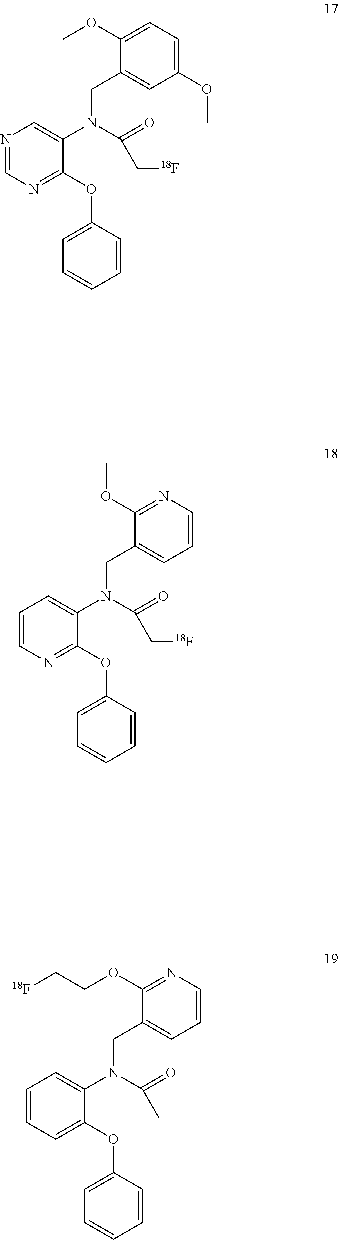

Preferred ¹⁸F-labelled in vivo imaging agents of the present invention are in vivo imaging agents 1, 18 and 19, most preferably in vivo imaging agent 1.

The potency (Example 6) of non-radioactive in vivo imaging agent 1 (illustrated above) was measured and compared with its isomer N-[3-(2-Fluoro-ethoxy)-benzyl[-N-(4-phenoxy-pyridin-3-yl)-acetamide (i.e. non-radioactive prior art compound [¹⁸F]-FE-PBR28). In an animal biodistribution model (Example 7), in vivo imaging agent 1 was tested and its biodistribution compared to that of the prior art compound [¹⁸F]-FE-PBR28 (prepared according to Wilson et al Nuc. Med. Biol. 2008; 35: 305-14).

Despite being an isomer of [¹⁸F]-FE-PBR28, the measured potency for PBR of imaging agent 1 was found to be two orders of magnitude less. Furthermore, an improved selectivity for PBR-expressing tissues in the brain was observed for imaging agent 1 compared with [¹⁸F]-FE-PBR28. The present inventors observed that the whole brain clearance ratio (2 min compared to 30 min) of [¹⁸F]-FE-PBR28 compared to imaging agent 1 is 1.97 compared to 3.46 showing that any imaging agent 1 unbound to the PBR receptor is more rapidly cleared from the brain. This is hypothesised to be a reason for the higher signal to background ratio observed for imaging agent 1 compared to [¹⁸F]-FE-PBR28.

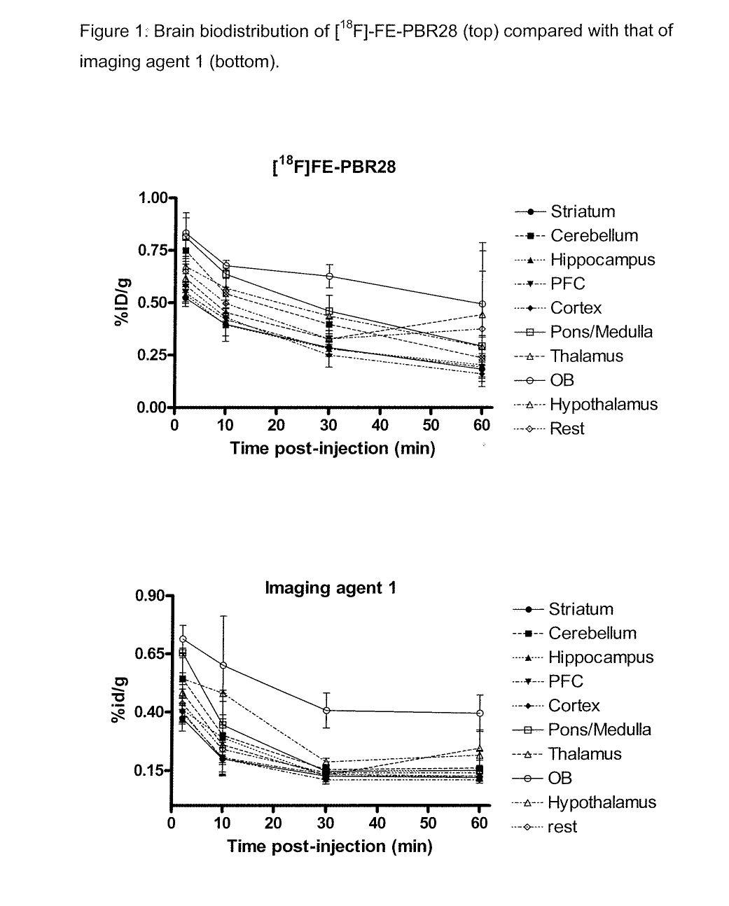

FIG. 1 shows the biodistribution of each compound in the brain up to 60 minutes post-injection. The graphs show that at 30 minutes post-injection, in vivo imaging agent 1 is retained more in the olfactory bulb (OB) as compared with the rest of the brain in comparison to [¹⁸F]-FE-PBR28, demonstrating that in vivo imaging agent 1 has improved selectivity for PBR than the previously-exemplified in vivo imaging agent, [¹⁸F]-FE-PBR28. Furthermore, the graphs illustrate that the clearance profile of imaging agent 1 is more favourable for in vivo imaging as compared with [¹⁸F]-FE-PBR28.

Method for Preparation

In a further aspect, the present invention provides a method for the preparation of the above-described in vivo imaging agent of the invention, said method comprising reaction of a suitable source of said radioisotope with a precursor compound of Formula II:

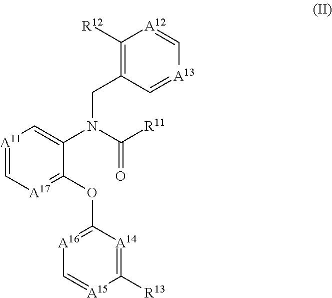

wherein:
one of R¹¹⁻¹³ comprises a precursor group; and, the rest of R¹¹⁻¹³ are as defined for R¹⁻³ of Formula I, respectively, and optionally comprise a protecting group;
A¹¹⁻¹⁷ are as defined for A¹⁻⁷ of Formula I, respectively, and optionally comprise a protecting group
with the proviso that when A¹⁷ is N and A¹¹, A¹² and A¹⁴⁻¹⁶ are all CH;
A¹³ is CH; and,
R¹³ is hydrogen.

A “precursor compound” comprises a non-radioactive derivative of a radiolabelled compound, designed so that chemical reaction with a convenient chemical form of said radioisotope suitable for in vivo imaging occurs site-specifically; can be conducted in the minimum number of steps (ideally a single step); and without the need for significant purification (ideally no further purification), to give the desired in vivo imaging agent. Such precursor compounds are synthetic and can conveniently be obtained in good chemical purity. The precursor compound may optionally comprise a protecting group for certain functional groups of the precursor compound. The precursor compound may be provided in solution in a kit, or in a cassette suitable for use with an automated synthesis apparatus, or alternatively attached to a solid support. The kit and cassette form additional aspects of the invention and will be discussed in more detail below.

By the term “protecting group” is meant a group which inhibits or suppresses undesirable chemical reactions, but which is designed to be sufficiently reactive that it may be cleaved from the functional group in question under mild enough conditions that do not modify the rest of the molecule. After deprotection the desired in vivo imaging agent is obtained. Protecting groups are well known to those skilled in the art and are described in ‘Protective Groups in Organic Synthesis’, Theodora W. Greene and Peter G. M. Wuts, (Third Edition, John Wiley & Sons, 1999).

The term “a suitable source of said radioisotope” means the radioisotope in a chemical form that is reactive with a substituent of the precursor compound such that the radioisotope becomes covalently attached to the precursor compound.

Broadly speaking, the step of “reacting” the precursor compound with the suitable source of said radioisotope involves bringing the two reactants together under reaction conditions suitable for formation of the desired in vivo imaging agent in as high a radiochemical yield (RCY) as possible. Some more detailed routes are presented in the experimental section below.

General methods to obtain a variety of in vivo imaging agents of the invention are now described. The skilled person would be able to apply the teachings described hereunder without any undue experimentation in order to obtain in vivo imaging agents over the entire scope of the present invention.

Okubu et at (2004 Bioorg. Med. Chem.; 12: 423-38) describe methods to obtain non-radioactive aryloxyanilide compounds. Synthetic schemes to obtain in vivo imaging agents similar to those of the present invention are described by Briard et al (J. Med. Chem. 2008; 51; 17-31), Wilson et al (Nuc. Med. Biol. 2008; 35; 305-14), and Zhang at al (J. Med. Chem. 2007; 50: 848-55). These prior art methods can be easily adapted to obtain precursor compounds suitable for obtaining in vivo imaging agents of the present invention.

Scheme I below is a generic reaction scheme to obtain precursor compounds suitable for preparation of the in vivo imaging agents of the present invention:

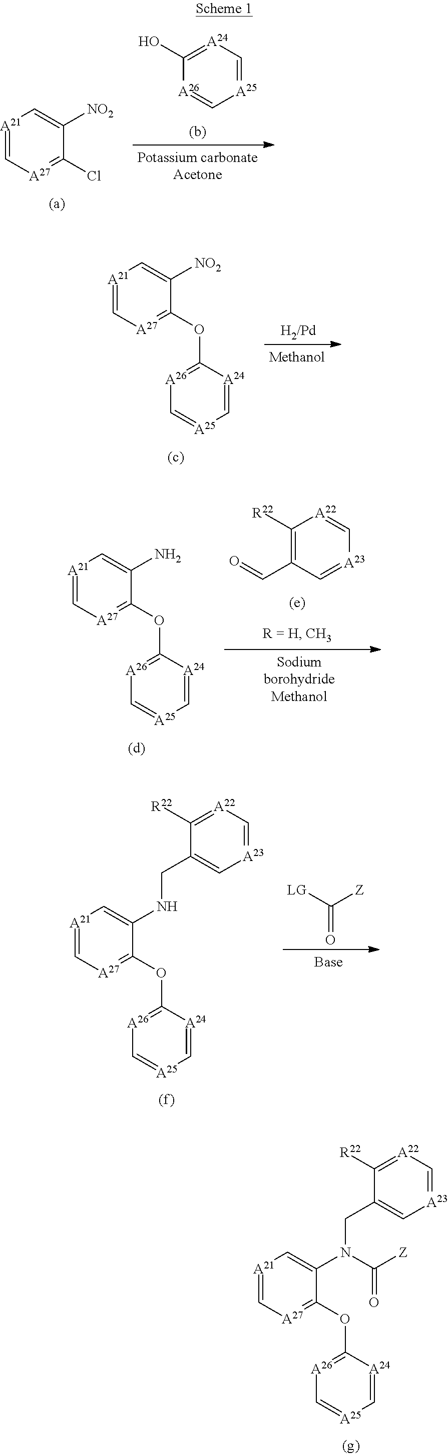

R²² is hydroxyl, alkoxy, or haloalkoxy, LG is a leaving group such as chloride or bromide, Z is (CH₂)ₓ—Y wherein x is 1-3, and Y is hydrogen or a group that can be displaced by a suitable source of a radioisotope suitable for in vivo imaging, and A²¹⁻²⁷ are as defined herein for A¹⁻⁷, respectively.

The ortho chloronitro aromatic (a) is reacted with a hydroxyl aromatic (b) under basic conditions when nucleophilic aromatic substitution occurs. Reduction of the nitro group by hydrogenation gives the corresponding aniline (d). Reductive alkylation with an aromatic aldehyde (e) gives the benzylamine (f). Acetylation gives the acetoxy amide (g). Depending on the radioisotope to be used for labelling, (g) itself may be a precursor compound, or may be converted into a precursor compound, as discussed further below.

When the radioisotope of the in vivo imaging agent is ¹⁸F, the radiofluorine atom may form part of a fluoroalkyl or fluoroalkoxy group, since alkyl fluorides are resistant to in vivo metabolism. Alternatively, the radiofluorine atom may be attached via a direct covalent bond to an aromatic ring.

Radiofluorination may be carried out via direct labelling using the reaction of ¹⁸F-fluoride with a suitable chemical group in the precursor compound having a good leaving group, such as an alkyl bromide, alkyl mesylate or alkyl tosylate. ¹⁸F can also be introduced by O-alkylation of hydroxyl groups with [¹⁸F]-fluoroalkyl bromide, [¹⁸F]-fluoroalkyl mesylate or [¹⁸F]-fluoroalkyl tosylate.

For aryl systems, ¹⁸F-fluoride nucleophilic displacement from an aryl diazonium salt, aryl nitro compound or an aryl quaternary ammonium salt are suitable routes to aryl-¹⁸F derivatives.

Alternatively, labelling with ¹⁸F can be achieved by nucleophilic displacement of a leaving group from a precursor compound. Suitable leaving groups include chloride, bromide, iodide, tosylate, mesylate, and triflate. Such derivatives are precursor compounds for the preparation of in vivo imaging compounds of the invention.

Another strategy would be to have a suitable leaving group as defined above in place on an alkylamide group present on the precursor compound. In this way, the precursor compound may be labelled in one step by reaction with a suitable source of [¹⁸F]-fluoride ion (¹⁸F⁻), which is normally obtained as an aqueous solution from the nuclear reaction ¹⁸O(p,n)¹⁸F and is made reactive by the addition of a cationic counterion and the subsequent removal of water. For this method, the precursor compounds are normally selectively chemically protected so that radiofluorination takes place at a particular site on the compound. Suitable protecting groups are those already mentioned previously.

When the radioisotope is ¹⁸F, it is preferred that the precursor compound comprises either:
(i) an alkyl halide or an alkyl sulfonate (such as alkyl bromide, alkyl mesylate or alkyl tosylate) for nucleophilic substitution; or,
(ii) hydroxyl (for introduction of ¹⁸F by O-alkylation of hydroxyl groups with e.g. ¹⁸F(CH₂)₃OMs or ¹⁸F(CH₂)₃Br).

In Scheme 2 below (which is a continuation of Scheme 1, above), Z is —(CH₂)— bromide or —(CH₂)-chloride. Treatment of this with [¹⁸F]-fluoride ion under basic conditions gives the labelled ¹⁸F fluoroacetyl compound (h), i.e. an in vivo imaging agent of Formula I wherein R¹ comprises ¹⁸F.

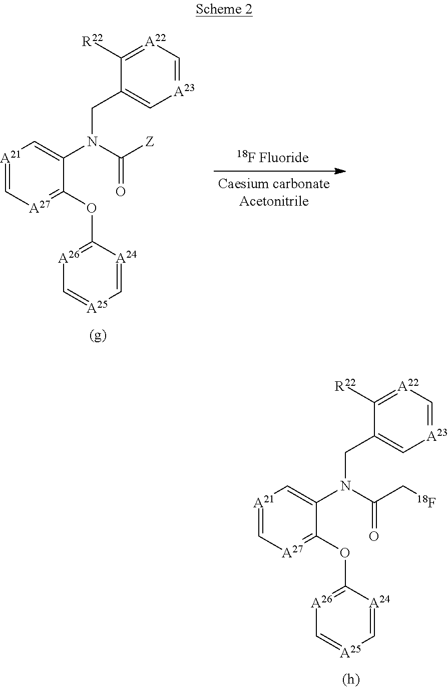

In Scheme 3 below (which is a continuation from (f) of Scheme 1, above), R²² of (f) is hydroxyl, and acetylation of (f) with acetyl-LG gives the acetoxy amide (i). Hydrolysis of the acetate gave the alcohol (j) that was alkylated with the ¹⁸F fluoroethyl tosylate and sodium hydride to give the ¹⁸F Fluoroethoxy compound (k), i.e. an in vivo imaging agent of Formula I wherein R² comprises ¹⁸F.

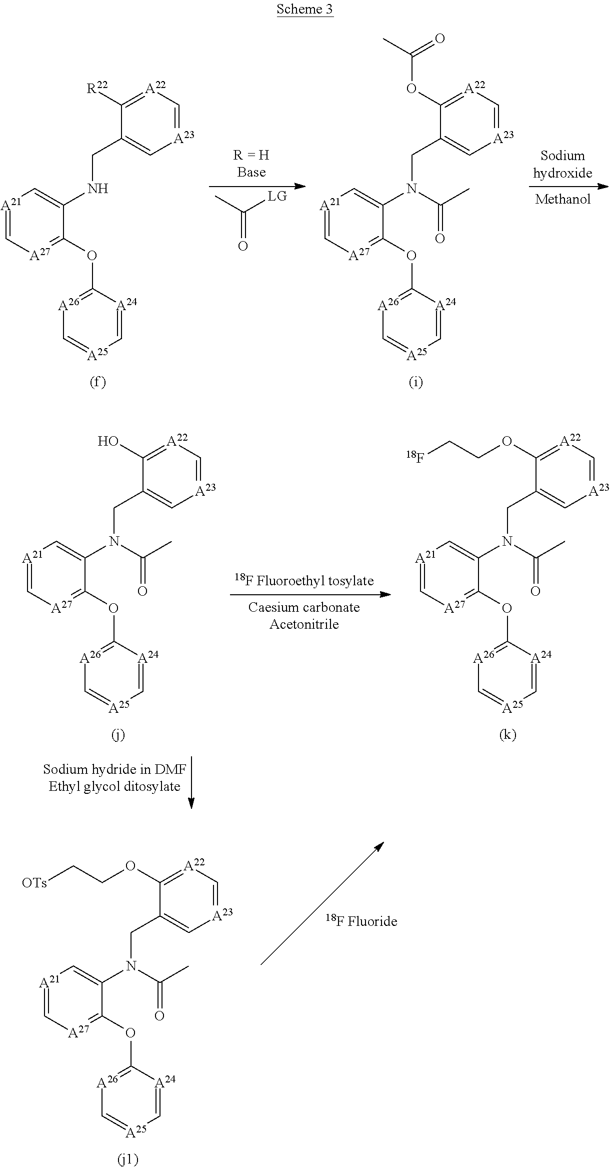

Imaging agent 1 can also obtained via this route:

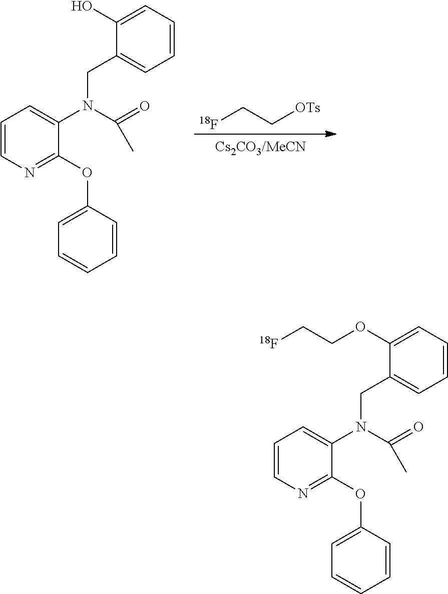

Still referring to Scheme 3, the same in vivo imaging agent (k) can be obtained by direct labelling with [¹⁸F]-Fluoride of a precursor compound comprising a leaving group. Such a direct labelling precursor where the leaving group is tosylate can be obtained by reaction of (j) with the particular alkyl glycol ditosylate to result in (j1). In turn, (j1) can be directly labelled with [¹⁸F]-Fluoride to obtain the ¹⁸F Fluoroethoxy compound (k). Imaging agent 1 can also obtained via this route:

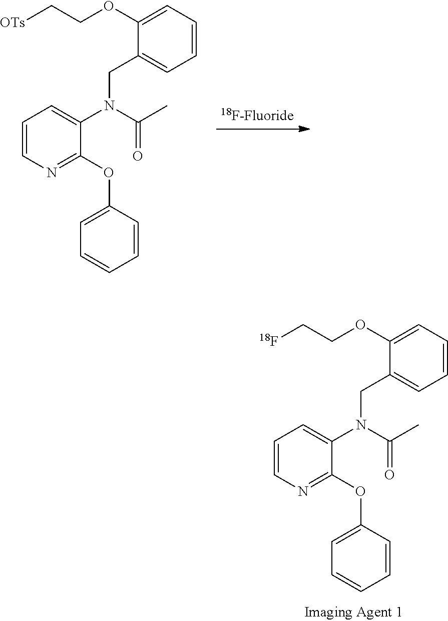

To obtain an in vivo imaging agent of the present invention where the radioisotope is radioiodine, a preferred precursor compound is one which comprises a derivative which either undergoes electrophilic or nucleophilic iodination or undergoes condensation with a labelled aldehyde or ketone. Examples of the first category are:
(a) organometallic derivatives such as a trialkylstannane (e.g. trimethylstannyl or tributylstannyl), or a trialkylsilane (e.g. trimethylsilyl) or an organoboron compound (e.g. boronate esters or organotrifluoroborates);
(b) aromatic rings activated towards electrophilic iodination (e.g. phenols) and aromatic rings activated towards nucleophilic iodination (e.g. aryl iodonium salt aryl diazonium, aryl trialkylammonium salts or nitroaryl derivatives).

For radioiodination, the precursor compound preferably comprises: an aryl iodide or bromide (to permit radioiodine exchange); an activated precursor compound aryl ring (e.g. a phenol group); an organometallic precursor compound (e.g. trialkyltin, trialkylsilyl or organoboron compound); or an organic precursor compound such as triazenes or a good leaving group for nucleophilic substitution such as an iodonium salt. Precursor compounds and methods of introducing radioiodine into organic molecules are described by Bolton (J. Lab. Comp. Radiopharm. 2002; 45: 485-528). Suitable boronate ester organoboron compounds and their preparation are described by Kabalka et at (Nucl. Med. Biol., 2002; 29: 841-843 and 2003; 30: 369-373). Suitable organotrifluoroborates and their preparation are described by Kabalka et al (Nucl. Med. Biol., 2004; 31: 935-938). Preferred precursor compounds for radioiodination comprise an organometallic precursor compound, most preferably a trialkyltin.

Examples of aryl groups to which radioactive iodine can be attached are given below:

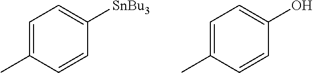

Both contain substituents which permit facile radioiodine substitution onto the aromatic ring. Alternative substituents containing radioactive iodine can be synthesised by direct iodination via radiohalogen exchange, e.g.

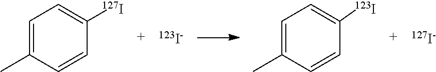

For example, in a precursor compound suitable for obtaining a radioiodinated in vivo imaging agent of the invention, either of R²¹ or R²³ of Formula III, together with the aromatic group to which it is attached, forms:
(i) an aromatic ring substituted with either an organometallic derivative or an organoboron compound;
(ii) an aromatic ring activated towards electrophilic radioiodination (e.g. phenols); or,
(iii) an aromatic ring activated towards nucleophilic radioiodination (e.g. aryl iodonium salt aryl diazonium, aryl trialkylammonium salts or nitroaryl derivatives).

These precursor compounds are easily converted into radioiodinated in vivo imaging agents of the invention by radioiodine substitution.

Radioiodinated compounds having a similar structure to the in vivo imaging agents of the present invention are described by Zhang et al (2007 J. Med. Chem.; 50: 848-55). These compounds were obtained by introduction of radioiodine onto a benzene ring by radioiodination of tributylstannane precursor compounds. An analogous method may be used to obtain radioiodinated in vivo imaging agents of the present invention, as illustrated in Scheme 4 below, where R²² of (g) is bromide (where Scheme 4 is a continuation of Scheme 1, above).

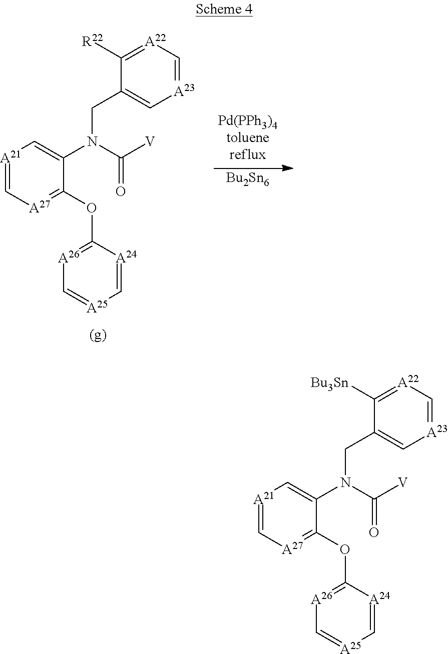

Compounds of Formula I where either R² or R³ are radiobromine can be obtained by radiobromination of the precursor compounds described above for radioiodinated compounds of Formula I. Kabalka and Varma have reviewed various methods for the synthesis of radiohalogenated compounds, including radiobrominated compounds (Tetrahedron 1989; 45(21): 6601-21).

A ¹¹C-labelled in vivo imaging agent of the invention may be synthesised in a straightforward manner by reacting a precursor compound which is a desmethylated version of the in vivo imaging agent with ¹¹C methyl iodide. Such a method is described by Briard et al (2008 J. Med. Chem.; 51 17-30) to obtain ¹¹C-labelled aryloxyanilide compounds. The methods described by Briard can be easily adapted using different starting materials to obtain ¹¹C labelled in vivo imaging agents of the present invention. Scheme 5 below (which is a continuation of Scheme 1, above), wherein R²² of (g) is hydroxyl, illustrates how the teachings of Briard et al may be adapted to obtain in vivo imaging agents of the present invention where the radioisotope is ¹¹C:

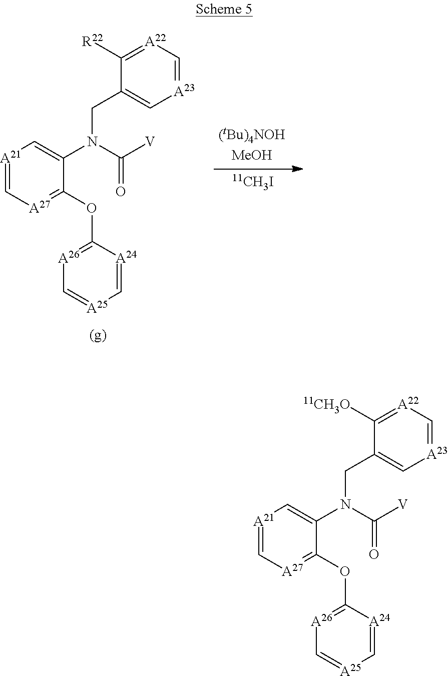

It is also possible to incorporate ¹¹C by reacting a Grignard reagent of the particular hydrocarbon of the desired in vivo imaging agent with [¹¹C]CO₂ to obtain a ¹¹C reagent that reacts with an amine group in the precursor compound to result in the ¹¹C-labelled in vivo imaging agent of interest. A Grignard reagent comprises a magnesium halide precursor group at the desired site of radiolabelling.

As the half-life of ¹¹C is only 20.4 minutes, it is important that the ¹¹C labelling intermediate has high specific activity and, consequently, that it is produced using a reaction process which is as rapid as possible. A thorough review of such ¹¹C-labelling techniques may be found in Antoni et al “Aspects on the Synthesis of ¹¹C-Labelled Compounds” in Handbook of Radiopharmaceuticals, Ed. M. J. Welch and C. S. Redvanly (2003, John Wiley and Sons).

The precursor compound of the invention is ideally provided in sterile, apyrogenic form. The precursor compound can accordingly be used for the preparation of a pharmaceutical composition comprising the in vivo imaging agent together with a biocompatible carrier suitable for mammalian administration. The precursor compound is also suitable for inclusion as a component in a kit for the preparation of such a pharmaceutical composition.

In a preferred embodiment, the precursor compound is provided in solution and as part of a kit or of a cassette designed for use in an automated synthesis apparatus. These aspects are discussed in more detail below in relation to additional aspects of the invention.

In another preferred embodiment, the precursor compound is bound to a solid phase. The precursor compound is preferably supplied covalently attached to a solid support matrix. In this way, the desired product forms in solution, whereas starting materials and impurities remain bound to the solid phase. As an example of such a system, precursor compounds for solid phase electrophilic fluorination with ¹⁸F-fluoride are described in WO 03/002489, and precursor compounds for solid phase nucleophilic fluorination with ¹⁸F-fluoride are described in WO 03/002157.

Most preferred precursor compounds for use in the method for preparation of the invention comprise precursor groups selected from alkyl bromide, alkyl mesylate, alkyl tosylate, a trialkylstannane, a trialkylsilane, or an organoboron compound. These most preferred precursor compounds themselves form a separate aspect of the present invention.

Radiopharmaceutical Composition

In another further aspect, the present invention provides a “radiopharmaceutical composition”, which is a composition comprising the in vivo imaging agent of the invention, together with a biocompatible carrier in a form suitable for mammalian administration.

The “biocompatible carrier” is a fluid, especially a liquid, in which the in vivo imaging agent is suspended or dissolved, such that the radiopharmaceutical composition is physiologically tolerable, i.e. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). The biocompatible carrier may also comprise biocompatible organic solvents such as ethanol. Such organic solvents are useful to solubilise more lipophilic compounds or formulations. Preferably the biocompatible carrier is pyrogen-free water for injection, isotonic saline or an aqueous ethanol solution. The pH of the biocompatible carrier for intravenous injection is suitably in the range 4.0 to 10.5.

Suitable and preferred embodiments of the in vivo imaging agent when comprised in the radiopharmaceutical composition of the invention are as already described herein.

The radiopharmaceutical composition may be administered parenterally, i.e. by injection, and is most preferably an aqueous solution. Such a composition may optionally contain further ingredients such as buffers; pharmaceutically acceptable solubilisers (e.g. cyclodextrins or surfactants such as Pluronic, Tween or phospholipids); pharmaceutically acceptable stabilisers or antioxidants (such as ascorbic acid, gentisic acid or para-aminobenzoic acid). Where the in vivo imaging agent of the invention is provided as a radiopharmaceutical composition, the method for preparation of said in vivo imaging agent may further comprise the steps required to obtain a radiopharmaceutical composition, e.g. removal of organic solvent, addition of a biocompatible buffer and any optional further ingredients. For parenteral administration, steps to ensure that the radiopharmaceutical composition is sterile and apyrogenic also need to be taken.

Kit and Cassette

In a preferred embodiment, the method for the preparation of the in vivo imaging agent of the invention is carried out by means of a kit, or using a cassette that can plug into an automated synthesiser. These kits and cassettes in turn form further aspects of the invention, and are particularly convenient for the preparation of the radiopharmceutical composition of the invention as defined herein.

The kit of the invention comprises the precursor compound of the invention in a sealed container. The “sealed container” preferably permits maintenance of sterile integrity and/or radioactive safety, plus optionally an inert headspace gas (e.g. nitrogen or argon), whilst permitting addition and withdrawal of solutions by syringe. A preferred sealed container is a septum-sealed vial, wherein the gas-tight closure is crimped on with an overseal (typically of aluminium). Such sealed containers have the additional advantage that the closure can withstand vacuum if desired e.g. to change the headspace gas or degas solutions.

Suitable and preferred embodiments of the precursor compound when employed in the kit of the invention are as already described herein.

The precursor compound for use in the kit may be employed under aseptic manufacture conditions to give the desired sterile, non-pyrogenic material. The precursor compound may alternatively be employed under non-sterile conditions, followed by terminal sterilisation using e.g. gamma-irradiation, autoclaving, dry heat or chemical treatment (e.g. with ethylene oxide). Preferably, the precursor compound is provided in sterile, non-pyrogenic form. Most preferably the sterile, non-pyrogenic precursor compound is provided in the sealed container as described above.

Preferably, all components of the kit are disposable to minimise the possibilities of contamination between runs and to ensure sterility and quality assurance.

In another aspect, the present invention provides a cassette which can be plugged into a suitably adapted automated synthesiser for the synthesis of the in vivo imaging agent of the invention. [¹⁸F]-radiotracers in particular are now often conveniently prepared on an automated radiosynthesis apparatus. There are several commercially-available examples of such apparatus, including Tracerlab™ and FASTlab™ (both available from GE Healthcare). The radiochemistry is performed on the automated synthesis apparatus by fitting the cassette to the apparatus. The cassette normally includes fluid pathways, a reaction vessel, and ports for receiving reagent vials as well as any solid-phase extraction cartridges used in post-radiosynthetic clean up steps.

The cassette for the automated synthesis of the in vivo imaging agent of the invention comprises:
(i) a vessel containing a precursor compound as defined herein; and
(ii) means for eluting the vessel with a suitable source of a radioisotope suitable for in vivo imaging, said radioisotope as defined herein.

The cassette may additionally comprise:
(iii) an ion-exchange cartridge for removal of excess said radioisotope; and optionally,
(iv) a cartridge for deprotection of the resultant radiolabelled product to form an in vivo imaging agent as defined herein.

The reagents, solvents and other consumables required for the automated synthesis may also be included together with a data medium, such as a compact disc carrying software, which allows the automated synthesiser to be operated in a way to meet the end user's requirements for concentration, volumes, time of delivery etc.

Methods of Use

In a yet further aspect, the present invention provides an in vivo imaging method for use in determining the distribution and/or the extent of PBR expression in a subject comprising:
(i) administering to said subject an in vivo imaging agent as defined herein;
(ii) allowing said in vivo imaging agent to bind to PBR in said subject;
(iii) detecting by an in vivo imaging procedure signals emitted by the radioisotope of said in vivo imaging agent;
(iv) generating an image representative of the location and/or amount of said signals; and,
(v) determining the distribution and extent of PBR expression in said subject wherein said expression is directly correlated with said signals emitted by said in vivo imaging agent.

For the in vivo imaging method of the invention, suitable and preferred aspects of the in vivo imaging agent are as defined earlier in the specification.

“Administering” the in vivo imaging agent is preferably carried out parenterally, and most preferably intravenously. The intravenous route represents the most efficient way to deliver the in vivo imaging agent throughout the body of the subject, and therefore also across the blood-brain barrier (BBB) and into contact with PBR expressed in said subject. The in vivo imaging agent of the invention is preferably administered as the pharmaceutical composition of the invention, as defined herein. The in vivo imaging method of the invention can also be understood as comprising the above-defined steps (ii)-(v) carried out on a subject to whom the in vivo imaging agent of the invention has been pre-administered

Following the administering step and preceding the detecting step, the in vivo imaging agent is allowed to bind to PBR. For example, when the subject is an intact mammal, the in vivo imaging agent will dynamically move through the mammal's body, coming into contact with various tissues therein. Once the in vivo imaging agent comes into contact with PBR, a specific interaction takes place such that clearance of the in vivo imaging agent from tissue with PBR takes longer than from tissue without, or with less PBR. A certain point in time will be reached when detection of in vivo imaging agent specifically bound to PBR is enabled as a result of the ratio between in vivo imaging agent bound to tissue with PBR versus that bound in tissue without, or with less PBR. Ideally, this ratio is 2:1 or greater.

The “detecting” step of the method of the invention involves detection of signals emitted by the radioisotope by means of a detector sensitive to said signals.

This detection step can also be understood as the acquisition of signal data. Single-photon emission tomography (SPECT) and positron-emission tomography (PET) are the most suitable in vivo imaging procedures for use in the method of the invention. PET is a preferred in vivo imaging procedure for use in the method of the invention.

The “generating” step of the method of the invention is carried out by a computer which applies a reconstruction algorithm to the acquired signal data to yield a dataset. This dataset is then manipulated to generate images showing the location and/or amount of signals emitted by said radioisotope. The signals emitted directly correlate with the expression of PBR such that the “determining” step can be made by evaluating the generated image.

The “subject” of the invention can be any human or animal subject. Preferably the subject of the invention is a mammal. Most preferably, said subject is an intact mammalian body in vivo. In an especially preferred embodiment, the subject of the invention is a human. The in vivo imaging method may be used to study PBR in healthy subjects, or in subjects known or suspected to have a pathological condition associated with abnormal expression of PBR (a “PBR condition”). Preferably, said method relates to the in vivo imaging of a subject known or suspected to have a PBR condition, and therefore is useful as part of a method for the diagnosis of said condition. Examples of such PBR conditions where in vivo imaging would be of use include neuropathologies such as Parkinson's disease, multiple sclerosis, Alzheimer's disease and Huntington's disease where neuroinflammation is present. Other PBR conditions that may be usefully imaged with the compounds of the invention include neuropathic pain, arthritis, asthma, atherosclerosis, as well as malignant diseases such as colorectal cancer and breast cancer. The in vivo imaging agents of the invention are particularly suited to in vivo imaging PBR expression in the central nervous system (CNS).

In an alternative embodiment, the in vivo imaging method of the invention may be carried out repeatedly during the course of a treatment regimen for said subject, said regimen comprising administration of a drug to combat a PBR condition. For example, the in vivo imaging method of the invention can be carried out before, during and after treatment with a drug to combat a PBR condition. In this way, the effect of said treatment can be monitored over time. Preferably for this embodiment, the in vivo imaging procedure is PET. PET has excellent sensitivity and resolution, so that even relatively small changes in a lesion can be observed over time, which is advantageous for treatment monitoring. PET scanners routinely measure radioactivity concentrations in the picomolar range. Micro-PET scanners now approach a spatial resolution of about 1 mm, and clinical scanners about 4-5 mm.

In a further aspect, the present invention provides a method for diagnosis of a PBR condition. The method of diagnosis of the invention comprises the in vivo imaging method as defined above, together with the further step (vi) of attributing the distribution and extent of PBR expression to a particular clinical picture, i.e. the deductive medical decision phase.

In another aspect, the present invention provides the in vivo imaging agent as defined herein for use in the method of diagnosis as defined herein.

In a yet further aspect, the present invention provides the in vivo imaging agent as defined herein for use in the manufacture of a radiopharmaceutical composition as defined herein for use in the method of diagnosis as defined herein.

The invention is now illustrated by a series of non-limiting examples.

### BRIEF DESCRIPTION OF THE EXAMPLES

Example 1 describes the synthesis of non-radioactive imaging agent 1.

Example 2 describes the synthesis of non-radioactive imaging agent 18.

Example 3 describes the synthesis of non-radioactive imaging agent 19.

Example 4 describes the synthesis of a direct labelling precursor compound for imaging agent 1.

Example 5 describes the synthesis of imaging agent 1.

Example 6 describes the in vitro potency assay used to determine binding affinity of imaging agents to PBR.

Example 7 describes the animal model used to determine biodistribution of imaging agents following intravenous administration.

### LIST OF ABBREVIATIONS USED IN THE EXAMPLES

° C. degrees celsius

aq aqueous

DMF dimethyl formamide

DMSO dimethyl sulfoxide

g grams

h hours

Kᵢ concentration of a compound required for half maximum inhibition

MBq megabequerels

mg milligrams

min minutes

ml millilitres

mM millimolar

mmol millimoles

n number of experiments

NMR nuclear magnetic resonance

PBR peripheral benzodiazepine receptor

RT room temperature

TLC thin layer chromatography

Tris tris(hydroxymethyl)aminomethane

UV ultraviolet

### EXAMPLES

### Example 1

### Synthesis of N-[2(2-fluoroethoxy)benzyl]-N-(2-phenoxypyridin-3-yl)acetamide (Non-Radioactive Imaging Agent 1)

### 1(i) 2-Phenoxy-3-nitropyridine

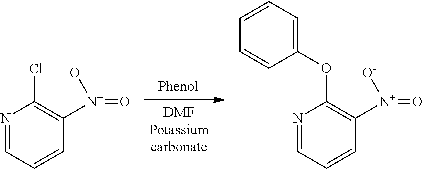

2-Chloro-3-nitropyridine (10 g, 63 mmol) in DMF (50 ml) was treated with phenol (8 g, 85 mmol) and potassium carbonate (15.4 g, 1.76 mmol) at 70° C. for 2 hr and then stirred at room temperature overnight. The reaction was then concentrated in high vacuum to a gum and diluted with into a mixture of ethyl acetate (50 ml), and water (150 ml) and stirred. The ethyl acetate solution was separated, dried over magnesium sulphate and concentrated in vacuum to a gum. The aqueous solution was re-extracted with a further 50 ml of ethyl acetate, the ethyl acetate layer was separated, dried over magnesium sulphate added to the previous ethyl acetate extract and concentrated in vacuum to give a yellow crystalline solid. The solid was washed with diethyl ether (20 ml) and collected by filtration to give colourless crystals of 2-phenoxy-3-nitropyridine (10.49 g, 46.5 mmol, 73.8%). The mother liquors were discarded.

¹H NMR (CDCl₃) 7.18(3H, m, ArH), 7.3(1H, m, ArH), 7.45(2H, t, ArH), 8.34(2H, m, ArH).

¹³C NMR (CDCl₃) 118.3, 121.6, 125.7, 129.6, 134.5, 135.4, 151.7, 152.5, 155.8.

### 1(ii) 2-phenoxy-3-aminopyridine

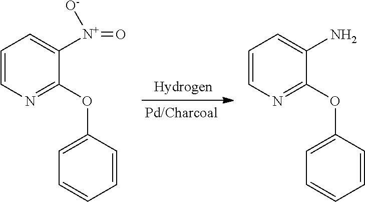

2-Phenoxy-3-nitropyridine (8.0 g, 37 mmol) in methanol (250 ml) was treated with palladium on charcoal (800 mg) under an atmosphere of hydrogen at 30° C. for 2 hr. There was a rapid uptake of hydrogen and a detectable exotherm with the temperature rising to 48° C. before dropping back. The reaction was then filtered through celite to give a colourless solution that was concentrated in high vacuum to give an oil 2-phenoxy-3-aminopyridine (6.8 g, 36 mmole, 98%) that crystallized on standing.

¹H NMR (CDCl₃) 3.96(2H, brs, NH₂), 6.86,(1H, m, ArH), 7.00(1H, m, ArH); 7.16(3H, m, ArH), 7.36(2H, m, ArH), 7.39(1H, m, ArH).

¹³C NMR (CDCl₃) 119.4, 120.6, 122.0, 124.2, 129.5, 131.9, 135.6, 151.6, 154.2.

### 1(iii) 2-[(2-Phenoxy-pyrid-3-ylamino)-methyl]-phenol

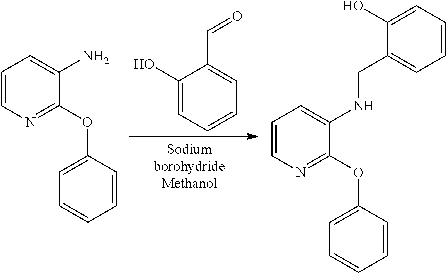

2-Phenoxy-3-aminopyridine (6 g, 32.25 mmol) was treated with o-salicyaldehyde (2-hydroxy-benzaldehyde) (6 g, 50 mmol) and toluene (10 ml) and heated at 90° C. for 1 h under an atmosphere of nitrogen with vigorous stirring. The solution became yellow and homogeneous. The reaction was then cooled to 0° C. when it solidified and was diluted with methanol (100 ml) when the solid all dissolved and treated with sodium borohydride (3.7 g, 97.5 mmol) in portions over a period of 20 min. A white precipitate formed over this period. The reaction was then allowed to warm to room temperature and stirred for a further 30 min. Formic acid (3 ml) was added and the reaction stirred for a further 18 h. A solid crystallized from the reaction and was collected by filtration (7.568 g) and dried in vacuum. The mother liquors were concentrated in vacuum to ˜30 ml and a further crop of crystals collected (2.2568 g). The solid was recrystallized from chloroform to give 2-[(2-phenoxy-pyrid-3-ylamino)methyl]-phenol (8.5 g, 29.1 mmol, 90%)

¹H NMR (CDCl₃) 1.69(1H, brs, NH); 4.45(2H, s, CH₂N); 4.75, (1H, brs, OH), 6.8-7.7, (12H, m, ArH).

¹³C NMR (CDCl₃) 46.9, 116.5, 119.3, 120.2, 120.4, 121.0, 121.5, 124.7, 128.8, 129.3, 129.6, 131.5, 133.0, 151.5, 152.0, 156.0.

### 1(iv) 2-[(Phenoxy-pyrid-3-ylacetylamino)-methyl]-phenolacetate

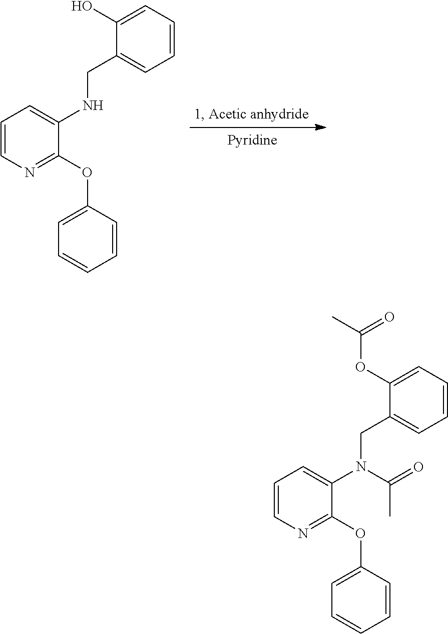

2-[(Phenoxy-pyrid-3-ylamino)-methyl-phenol (1 g, 3.42 mmol) in dichloromethane (10 ml) was treated with acetic anhydride (1.39 g, 13.6 mmol) and pyridine (1.074 g, 13.6 mmol) and stirred at 20° C. for 18 h under an atmosphere of nitrogen. The reaction was then diluted with dichloromethane (50 ml) and washed with 5N hydrochloric acid (20 ml) to remove the pyridine and the organic layer separated dried over magnesium sulphate and concentrated in high vacuum to a gum that crystallized on standing. Recrystallization from diethyl ether and petrol ether to give a white solid 2-[(Phenoxy-pyrid-3-ylacetylamino)methyl-phenolacetate (1.05 g, 2.79 mmol, 81%).

¹H NMR (CDCl₃) 1.93(3H, s, CH₃), 2.25 (3H, s, CH₃), 4.37(2H, d, CH), and 5.54(2H, d, CH), together CH₂N, 6.5-7.5 (10H, m, ArH), 8.0(1H, d, ArH).

¹³C NMR (CDCl₃) 20.9, 22.1, 45.4, 118.8, 121.2, 122.7, 125.1, 125.7, 125.9, 128.8, 129.0, 129.6, 131.9, 140.0, 147.0, 1249.5, 153.1, 159.4, 169.7, 170.1

### 1(v) N-(2-hydroxybenzyl)-N-(phenoxy-pyrid-3-yl)-acetamide

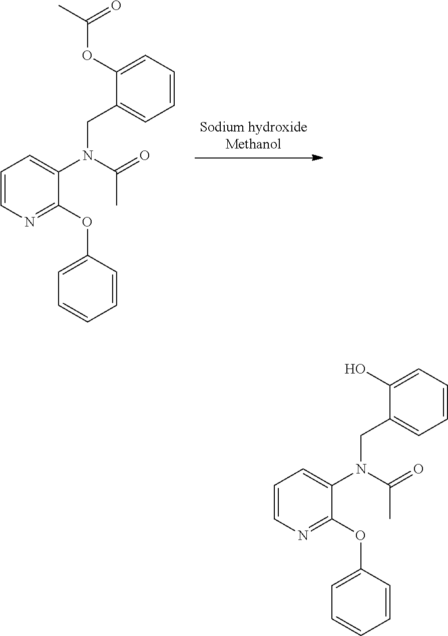

N-(2-Acetoxybenzyl)-N-(phenoxy-pyrid-3-yl)-acetamide (0.5 g, 1.71 mmol) in methanol (20 ml) and treated with sodium hydroxide (212 mg, 5.3 mmol) and stirred at room temperature for 30 min. TLC run in 20% ethyl acetate in dichloromethane on silica showed complete conversion of the acetate to the (surprisingly) faster running phenol. The reaction was then adjusted to neutrality (monitored by litmus paper) with acetic acid (˜318 mg, 5.3 mmol) and concentrated in vacuum to give a white solid. The solid was partitioned between dichloromethane (50 ml) and water (50 ml). The dichloromethane layer was separated dried over magnesium sulphate and concentrated in vacuum to a solid N-(2-hydroxybenzyl)-N-(phenoxy-pyrid-3-yl)-acetamide (411 mg, 1.22 mmol, 91%).

¹H NMR (CDCl₃) 2.01(3H, s, CH₃), 4.63(1H, s), 5.02(1H,s) together CH₂N, 6.6-7.4 (11H, m, ArH), 8.17(1H, d, ArH), 9.34(1H, s, OH).

¹³C NMR (CDCl₃) 21.7, 49.7, 117.7, 118.9, 119.25, 121.3, 121.6, 125.3, 125.5, 129.6, 130.3, 131.3, 138.8. 147.7, 152.7, 156.1, 159.1, 173.4.

### 1(vi) N-[2(2-fluoroethoxy)benzyl]-N-(2-phenoxypyridin-3-yl)acetamide

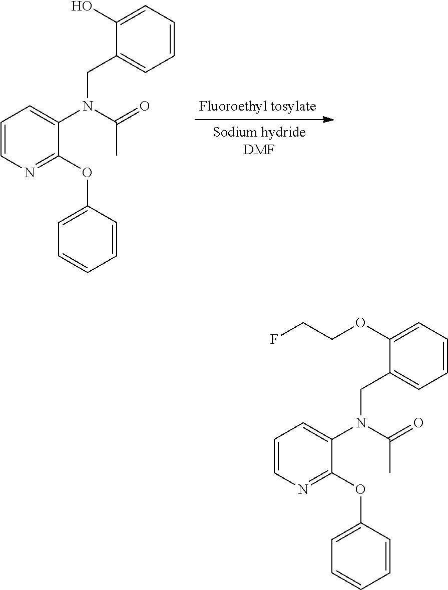

N-[2(2-hydroxy)benzyl]-N-(2-phenoxypyridin-3-yl)acetamide (300 mg, 0.898 mmol) in DMF (10 ml) was treated with sodium hydride (96 mg, 2.4 mmol) and 2-fluoroethyltosylate (527 g, 2.4 mmol) and stirred at 30° C. for 1 h under an atmosphere of nitrogen. The reaction was monitored by TLC run in 20% ethyl acetate in dichloromethane visualised under UV light. This showed the formation of a slower running spot that was complete after 1 h. The reaction was then quenched by the addition of acetic acid (1 ml) and concentrated in high vacuum to give an oil. The oil was partitioned between ethyl acetate (100 ml) and sodium bicarbonate (50 ml) solution. The ethyl acetate solution was separated dried over magnesium sulphate and concentrated in vacuum to a gum. The gum was chromatographed on silica in a gradient of 5-20% ethyl acetate in dichloromethane to give two fractions. Fraction 1 was recovered fluoroethyltosylate eluting essentially in the void volume and fraction 2 eluting after about 6 column volumes was N-[2(2-fluoroethoxy)benzyl]-N-(2-phenoxypyridin-3-yl) acetamide (332 mg, 0.87 mmole, 97%).

¹H NMR (CDCl₃) 1.98(3H, s, CH₃), 3.67(3H, s, OCH₃),3.9(2H, brm, CH₂O), 4.44, and 4.60(2H, each m, CH₂F), 4.86(1H, d), and 5.23(1H, d) together CH₂N, 6.67-7.37(10H, m, ArH), 8.0(1h, s, ArH)

¹³C NMR (CDCl₃) 22.3, 45.6, 55.6, 67.9, 68.1, 80.7, 83.0, 112.8, 113.7, 116.7, 118.6, 121.3, 125.0, 126.7, 129.5, 139.1, 146.6, 150.5, 153,2, 153.9, 159.6, 170.5.

### Example 2

### Synthesis of N-[2-(2-Fluoro-ethoxy)-pyridin-3-ylmethyl]-N-(2-phenoxy-phenyl)-acetamide (Non-Radioactive Imaging Agent 19)

### 2(i) 2-aminodiphenyl ether

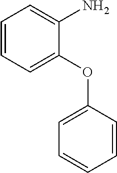

2-Nitrodiphenyl ether (16 g, 74 mmol) in methanol (250 ml) was shaken with palladium on charcoal (1.6 g) under an atmosphere of hydrogen at 20-50° C. for 30 min. There was a rapid uptake of hydrogen and a detectable exotherm 20-50° C. with the temperature rapidly rising before finally dropping back. Shaking was stopped for short periods to control the temperature from rising above 50° C. The reaction was then filtered through celite and concentrated in high vacuum to give 2-aminodiphenyl ether (13.5 g, 72.9 mmole, 98%) as an oil that crystallized on standing.

¹H NMR (300 MHz, CDCl₃): δ_{H} 3.8(2H, brs,NH), 6.7-6.75(1H, m, ArH), 6.8-6.94(2H, m, ArH), 6.94-7.1 ((4H, m, ArH), 7.25-7.4(2H, m, ArH).

¹³C NMR (75 MHz, CDCl₃): δ_{c} 116.4, 117.1, 118.7, 120.2, 12206, 124.9, 129.7, 138.7, 143.0, 157.5.

### 2(ii) (2-Methoxy-pyridin-3-ylmethyl)-(2-phenoxy-phenyl)-amine

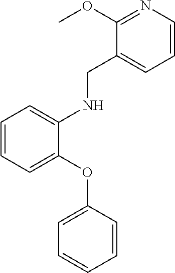

A mixture of 2-aminodiphenyl ether from step 2(i) (1.80 g, 9.8 mmol) and 2-methoxy-3-pyridinecarboxaldehyde (2.0 g, 14.6 mmol) was heated at 90° C. for 1 h under nitrogen. The reaction mixture was cooled to 0° C. and MeOH (20 mL) was added, followed by sodium borohydride (1.11 g, 29.4 mmol) in portions over 20 min. The mixture was stirred at RT for 24 h. Formic acid (2.4 g, 53.0 mmol, 2.0 mL) was added and the mixture stirred for 15 min. The solvents were removed in vacuo, the residue quenched with 10% aqueous sodium bicarbonate (100 mL), extracted with DCM (2×30 mL), dried over magnesium sulfate, filtered and solvents removed in vacuo. The crude material was purified by silica gel chromatography eluting with DCM (A): ethyl acetate (B) (2% (B), 80 g, 2.0 CV, 60 mL/min) to afford impure product. The sample was crystallized from methanol to afford 2.2 g (73%) of (2-Methoxy-pyridin-3-ylmethyl)-(2-phenoxy-phenyl)-amine as a white solid.

¹H NMR (300 MHz, CDCl₃): δ_{H} 3.90 (3H, s, OCH₃), 4.33 (2H, d, J=6 Hz, NCH₂), 4.73 (1H, m, NH), 6.61-6.68 (2H, m, Ph), 6.77-7.09 (6H, m, Ph), 7.27-7.34 (2H, m, Ph), 7.48 (1H, m, Ph), and 8.03 (1H, dd, J=2 and 5 Hz, Ph).

LC-MS: m/z calcd for C₁₉H₁₈N₂O₂, 306.1; found, 307.1 (M+H)⁺.

### 2(iii) N-(2-Methoxy-pyridin-3-ylmethyl)-N-(2-phenoxy-phenyl)-acetamide

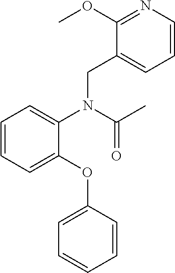

To a solution of (2-Methoxy-pyridin-3-ylmethyl)-(2-phenoxy-phenyl)-amine from step 2(ii) (1.0 g, 3.26 mmol) dissolved in anhydrous DCM (15 mL) was added 4-(Dimethylamino)pyridine (0.01 g, 0.08 mmol). The reaction was cooled to 0° C. and acetyl chloride (1.54 g, 19.6 mmol, 1.40 mL) was added. The mixture was stirred at RT for 3 h. The solvents were removed in vacuo, the residue quenched with 1N aqueous sodium hydroxide (5 mL), extracted with DCM (2×20 mL), dried over magnesium sulfate, filtered and solvents removed in vacuo. The crude material was purified by silica gel chromatography eluting with DCM (A): methanol (B) (5% (B), 80 g, 2.0 CV, 60 mL/min) to afford 1.1 g (97%) of N-(2-Methoxy-pyridin-3-ylmethyl)-N-(2-phenoxy-phenyl)acetamide as a colourless oil.

¹H NMR (300 MHz, CDCl₃): δ_{H} 1.97 (3H, s, COCH₃), 3.74 (3H, s, OCH₃), 4.73 (1H, d, J=15 Hz, NCH), 4.99 (1H, d, J=15 Hz, NCH), 6.72-6.91 (4H, m, Ph), 6.98-7.37 (6H, m, Ph), 7.65 (1H, dd, J=2 and 7 Hz, Ph), and 8.00 (1H, dd, J=2 and 5 Hz, Ph).

### 2(iv) 3-[(2-Phenoxy-phenylamino)-methyl]-pyridin-2-ol

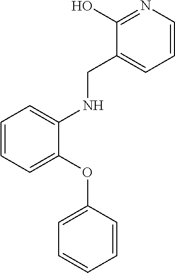

A solution of N-(2-Methoxy-pyridin-3-ylmethyl)-N-(2-phenoxy-phenyl)-acetamide from step 2(iii) (0.60 g, 1.72 mmol) in 48% aqueous hydrobromic acid (44.7 g, 552.0 mmol, 30 mL) was heated at 100° C. for 24 h under nitrogen. The solvents were removed in vacuo, the residue quenched with 10% aqueous potassium carbonate (50 mL), extracted with DCM (2×30 mL), dried over magnesium sulfate, filtered and solvents removed in vacuo to afford 0.51 g (100%) of 3-[(2-Phenoxy-phenylamino)-methyl]-pyridin-2-ol as a gum.

¹H NMR (300 MHz, CDCl₃): δ_{H} 4.32 (2H, s, NCH₂), 4.90 (1H, s, br, NH), 6.22 (1H, t, J=7 Hz, Ph), and 6.60-7.44 (12H, m, Ph).

LC-MS: m/z calcd for C₁₈H₁₆N₂O₂, 292.1; found, 293.1 (M+H)⁺.

### 2(v) N-(2-Hydroxy-pyridin-3-ylmethyl)-N-(2-phenoxy-phenyl)-acetamide

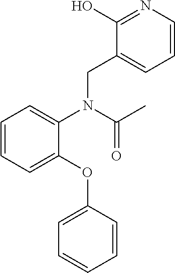

To a solution of 3-[(2-Phenoxy-phenylamino)-methyl]-pyridin-2-ol from step 2(iv) (0.51 g, 1.74 mmol) dissolved in anhydrous DCM (20 mL) was added 4-(Dimethylamino)pyridine (0.01 g, 0.08 mmol). The reaction was cooled to 0° C. and acetyl chloride (1.6 g, 20.8 mmol, 1.5 mL) was added. The mixture was stirred at RT for 24 h. The solvents were removed in vacuo and saturated lithium hydroxide in methanol (10 mL) was added to the residue. After stirring for 10 min, water (20 mL) was added and methanol removed in vacuo. The aqueous solution was extracted with DCM (2×20 mL), the combined organics washed with brine (20 mL), dried over magnesium sulfate, filtered and solvents removed in vacuo. The crude material was purified by silica gel chromatography eluting with DCM (A): methanol (B) (10% (B), 12 g, 1.0 CV, 30 mL/min) to afford impure product. The sample was repurified by silica gel chromatography eluting with DCM (A): methanol (B) (2-10% (B), 40 g, 8.0 CV, 40 mL/min) to afford 0.35 g (59%) of N-(2-Hydroxy-pyridin-3-ylmethyl)-N-(2-phenoxy-phenyl)-acetamide as a white foam.

¹H NMR (300 MHz, CDCl₃): δ_{H} 2.01 (3H, s, COCH₃), 4.75 (1H, d, J=16 Hz, NCH), 4.88 (1H, d, J=16 Hz, NCH), 6.12 (1H, t, J=7 Hz, Ph), 6.80-7.38 (10H, m, Ph), 7.62 (1H, d, J=7 Hz, Ph), and 12.66 (1H, s, br, OH).

LC-MS: m/z calcd for C₂₀H₁₈N₂O₃, 334.1; found, 357.0 (M+Na)⁺.

### 2(vi) Non-Radioactive In Vivo Imaging Agent 19

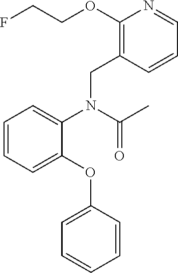

N-(2-Hydroxy-pyridin-3-ylmethyl)-N-(2-phenoxy-phenyl)acetamide from step 2(v) (0.15 g, 0.45 mmol) was dissolved in anhydrous DMF (2 mL) at RT under nitrogen. Potassium carbonate (0.19 g, 1.35 mmol) and 2-fluoroethyl tosylate (0.20 g, 0.89 mmol) were added and the mixture heated at 70° C. for 24 h. The DMF was removed in vacuo, the residue quenched with water (40 mL), extracted with DCM (2×20 mL), dried over magnesium sulfate, filtered and solvents removed in vacuo. The crude material was purified by silica gel chromatography eluting with DCM (A): methanol (B) (2-5% (B), 40 g, 3.0 CV and 7.0 CV, 40 mL/min) to afford impure O-alkyl and N-alkyl products. The O-alkyl sample was repurified by silica gel chromatography eluting with DCM (A): methanol (B) (1% (B), 40 g, 4.0 CV, 40 mL/min) to afford impure product. The sample was repurified by silica gel chromatography eluting with DCM (A): ethyl acetate (B) (10-90% (B), 40 g, 18.0 CV, 40 mL/min) to afford 35 mg (20%) of non-radioactive in vivo imaging agent 19 as a colourless oil.

¹H NMR (300 MHz, CDCl₃): δ_{H} 1.96 (3H, s, COCH₃), 4.24-4.65 (4H, m, OCH₂CH₂F), 4.74 (1H, d, J=15 Hz, NCH), 5.05 (1H, d, J=15 Hz, NCH), 6.75-7.36 (10H, m, Ph), 7.70 (1H, dd, J=2 and 7 Hz, Ph), and 7.97 (1H, dd, J=2 and 5 Hz, Ph).

LC-MS: m/z calcd for C₂₂H₂₁FN₂O₃, 380.2; found, 381.1 (M+H)⁺.

### Example 3

### Synthesis of 2-Flouro-N-(2-methoxy-pyridin-3-ylmethyl)-N-(2-phenoxy-phenyl)-acetamide (Non-Radioactive In Vivo Imaging Agent 18)

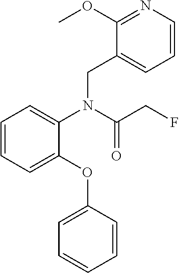

To a solution of N-(2-Methoxy-pyridin-3-ylmethyl)-N-(2-phenoxy-phenyl)-acetamide as obtained in step 2(iii) (0.31 g, 1.0 mmol) dissolved in anhydrous DCM (5 mL) was added 4-(Dimethylamino)pyridine (0.01 g, 0.08 mmol). The reaction was cooled to 0° C. and fluoroacetyl chloride (0.58 g, 6.0 mmol, 0.40 mL) was added. The mixture was stirred at RT for 3 h. The solvents were removed in vacuo, the residue quenched with 1N aqueous sodium hydroxide (5 mL), extracted with DCM (2×20 mL), dried over magnesium sulfate, filtered and solvents removed in vacuo. The crude material was purified by silica gel chromatography eluting with DCM (A): methanol (B) (1-5% (B), 80 g, 6.0 CV, 60 mL/min) to afford 0.26 g (71%) of non-radioactive imaging agent 18 as a white solid.

¹H NMR (300 MHz, CDCl₃): δ_{H} 3.72 (3H, s, OCH₃), 4.69 (1H, s, FCH), 4.79 (1H, d, J=15 Hz, NCH), 4.84 (1H, s, FCH), 5.02 (1H, d, J=15 Hz, NCH), 6.72-6.88 (4H, m, Ph), 6.98-7.38 (6H, m, Ph), 7.64 (1H, dd, J=2 and 7 Hz, Ph), and 8.02 (1H, dd, J=2 and 5 Hz, Ph); ¹⁹F NMR (283 MHz, CDCl₃): δ_{F}-226.9.

LC-MS: m/z calcd for C₂₁H₁₉FN₂O₃, 366.1, found, 367.1 (M+H)⁺.

### Example 4

### Synthesis of 2-[(Phenoxy-pyrid-3-ylacetylamino)-methylphenol)2-ethyl toluene-4-sulphonate (Direct Labelling Precursor Compound for Imaging Agent 1)

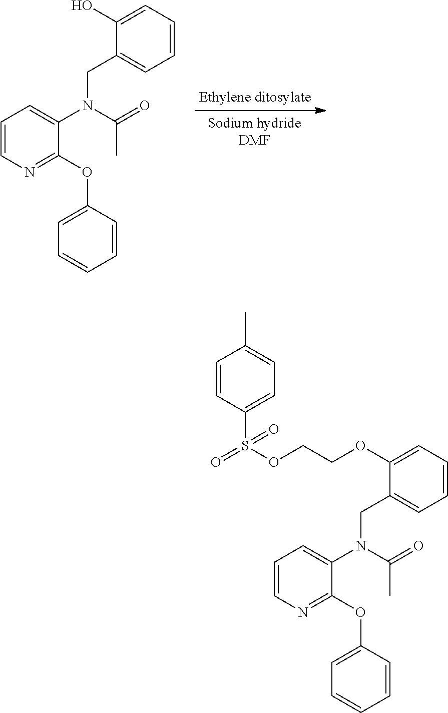

2-[(Phenoxy-pyrid-3-ylacetylamino)-methyl-phenol (1 g, 3.0 mmol) in DMF (30 ml) was treated with sodium hydride (60%) (478 mg, 2.4 mmol) and ethyl glycol ditosylate (2.77 g, 7.5 mmol) and stirred at 20° C. for 1 h under an atmosphere of nitrogen. The reaction was monitored by TLC run in 50% ethyl acetate in petrol visualised under UV light. This showed disappearance of starting material and appearance of a slower running material. The excess Ethylene glycol ditosylate ran as a spot slightly faster running than the starting material. After 1 h the reaction was complete by TLC. The reaction was then quenched by the dropwise addition of acetic acid (450 mg) when there was a vigorous evolution of hydrogen gas. The reaction was then concentrated in high vacuum to give an oil. The oil was partitioned between ethyl acetate (100 ml) and sodium bicarbonate (50 ml) solution. The ethyl acetate solution was separated dried over magnesium sulphate and concentrated in vacuum to a gum. Some of the excess ethyl glycol ditosylate crystallized out and was separated by trituration with ether and filtration. The gum was chromatographed on silica in a gradient of 5-20% ethyl acetate in petrol to give three fractions. Fraction 1 was recovered ethylene glycol ditosylate Fraction 2 was a small amount of recovered starting material and fraction 3 was the required compound (1.246 g, 2.34 mmole, 78%).

¹H NMR (CDCl₃) δ, 2.0(3H, s, CH₃), 2.45, (3H, s, CH₃), 3.9-4.2, (4H, m, 2CH₂), 4.87 and 5.04(1H, d, together CH₂N), 6.62 (1H, d, ArH), 6.8-6.95, (4H, m, ArH), 7.1-7.54(8H, m, ArH), 7.77((2H, d, ArH), 8.0(1H, d, ArH).

¹³C NMR CDCl₃ δ, 21.49, 22.18, 45.7, 65.3, 67.9, 110.9, 118.6, 121.2, 124.9, 125.1, 126.4, 127.7, 128.7129.4, 129.8, 131 1, 132.6, 139, 145.0, 146.4, 153.0, 155.8, 159.4, 170.4.

### Example 5

### Indirect Labelling Synthesis of Imaging Agent 1

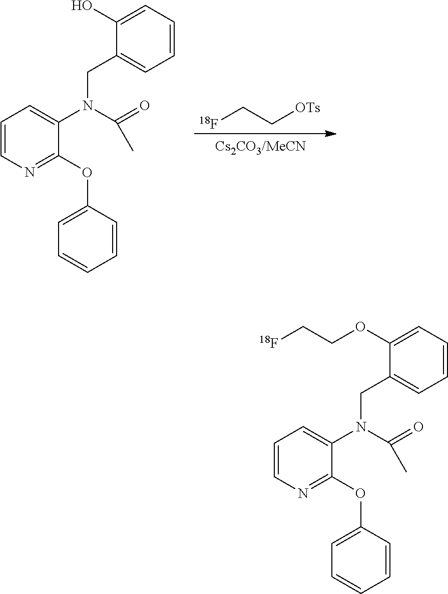

A mixture of Kryptofix 2.2.2 (4 mg, 10.6 μmol), potassium carbonate (0.1 mol dm⁻³, 50 μl, 0.7 mg, 5 μmol) and acetonitrile (0.5 mL) was added to [¹⁸F]F⁻/H₂O (ca. 400 MBq, 100-300 μl) in a reaction vessel The solvent was removed by heating at 100° C. under a stream of nitrogen for 15-20 minutes. A solution of ethylene glycol-ditosylate (3-5 mg, 8-13.5 μmol) in acetonitrile (1 mL) was added and the mixture was heated at 100° C. for 10 minutes. After cooling, the reaction was removed; the reaction vessel was rinsed with water (1000 μl) and added to the main crude reaction. [¹⁸F]fluoroethyltosylate was purified by HPLC (ACE C18(2) column, 5 u, 100×10 mm, 5 ml loop, pump speed 3 ml/min, wavelength 254 nm, mobile phase water:MeOH: 0-1 min 50% MeOH; 1-25 min 50-95% MeOH; 25-30 min 95% MeOH; 30-31 min 95-50% MeOH; 31-33 min 50% MeOH). t_{R} [¹⁸F]fluoroethyltosylate 7.5 min. Radiochemical yield of [¹⁸F]fluoroethyltosylate, ca. 40% non decay corrected yield.

The [¹⁸F]fluoroethyltosylate cut peak was diluted to a volume of ca. 20 ml with H₂O, loaded onto a conditioned light t-C18 sep pak and flushed with H₂O (1×2 ml). The loaded sep pak was dried on a high flow N₂ line for 15-20 mins.

A Wheaton vial (1 ml) containing a stirrer, N-(2-hydroxybenzyl)-N-(phenoxy-pyrid-3-yl)-acetamide (2-4 mg, 6-12 μmol, prepared according to Example 1(v)), Cs₂CO₃ (8-10 mg, 24-30 μmol)) in CH₃CN(100 μl) was stirred at room temperature for 1-2 h. The activity on the dried lite t-C18 sep pak was eluted with CH₃CN (0.5 ml) into the Wheaton vial. The Wheaton vial was sealed, the reaction was heated and stirred in an oil bath at 120-130° C./15 mins. After, the reaction was cooled and quenched with water (500 μl). Imaging agent 1 was purified by HPLC (ACE C18(2) column, 5 u, 100×10 mm, 5 ml loop, pump speed 3 ml/min, wavelength 254 nm, mobile phase water:MeOH: 0-1 min 50% MeOH; 1-20 min 50-95% MeOH; 20-25 min 95% MeOH; 25-26 min 95-50% MeOH; 26-28 min 50% MeOH).

### Example 6

### In Vitro Potency Assay

Affinity for PBR was screened using a method adapted from Le Fur et al (Life Sci. 1983; USA 33: 449-57). The compounds tested were non-radioactive imaging agent 1 and the non-radioactive version of the prior art compound FE-PBR28.

Each test compound (dissolved in 50 mM Tris-HCl, pH 7.4, 10 mM MgCl₂ containing 1% DMSO) competed for binding to Wistar rat heart PBR against 0.3 nM [³H] PK-11195. The reaction was carried out in 50 mM Tris-HCl, pH 7.4 10 mM MgCl₂ for 15 minutes at 25° C.

Each test compound was screened at 6 different concentrations over a 300-fold range of concentrations around the estimated Kᵢ. The Kᵢ for non-radioactive imaging agent 1 was found to be 4.24 nM and for FE-PBR28 was found to be 0.056 nM.

| Compound | Ki (nM) |
|---|---|
| FE-PBR28 | 0.056 |
| Non-radioactive imaging agent 1 | 4.24 |
| Non-radioactive imaging agent 18 | 3.77 |
| Non-radioactive imaging agent 19 | 1.02 |

### Example 7

### In Vivo Biodistribution Method

In vivo imaging agent 1 and the prior art compound [¹⁸F]-FE-PBR28 (prepared according to Wilson et al Nuc. Med. Biol. 2008; 35: 305-14) were tested in the in vivo biodistribution model and their respective biodistributions compared

Adult male Wistar rats (200-300 g) were injected with 1-3 MBq of in vivo imaging agent via the lateral tail vein. At 2, 10, 30 or 60 min (n=3) after injection, rats were euthanised and tissues or fluids were sampled for radioactive measurement on a gamma counter. FIG. 1 illustrates the data obtained. More specifically, following injection of imaging agent, uptake in the olfactory bulb (OB) 2 minutes post-injection was examined, as well as the ratio of uptake in OB to uptake in the striatum at 30 minutes post-injection. The rationale for these specific measurements is that the OB is known to express higher levels of PBR compared with other areas of rat brain (see “Handbook of Substance Abuse” by Tarter, Ammerman and Ott; Springer 1998: 398-99).

Table 1 below compares imaging agent 1 alongside [¹⁸F]-FE-PBR28:

| | OB* uptake | Selectivity for PBR |
|---|---|---|
| Imaging Agent | (ID/g* @2 min) | (OB*: Striatum @30 min) |
| Imaging Agent 1 | 0.71 | 3.08 |
| [¹⁸F]-FE- PBR28 | 0.83 | 2.17 |

*ID/g = injected dose per gram; OB = olfactory bulb

What is claimed is:

1) An in vivo imaging agent of Formula I: 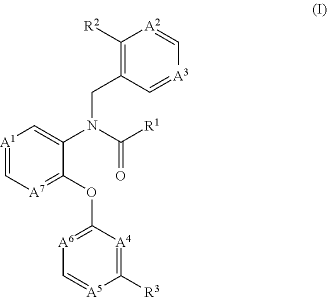
or a salt or solvate thereof, wherein:
R¹ is selected from methyl and C₁₋₃ fluoroalkyl;
R² is selected from hydrogen, halogen, C₁₋₃ alkoxy, and C₁₋₃ fluoroalkoxy;
R³ is selected from hydrogen, halogen and C₁₋₃ alkoxy;
A¹ is N or CH;
1 or 2 of A² and A⁴⁻⁷ are N, and the rest of A² and A⁴⁻⁷ are CH;
A³ is CH or CH—O—R⁴, where R⁴ is H, C₁₋₃ alkyl or C₁₋₃ haloalkyl, or A³ can alternatively be N when one of A² and A⁴⁻⁷ is N, and the rest of A² and A⁴⁻⁷ are CH;
and wherein the agent of Formula I comprises an atom which is a radioisotope suitable for in vivo imaging.

2) The in vivo imaging agent as defined in claim 1 wherein R¹ is C₁₋₃ fluoroalkyl and R² is hydrogen.

3) The in vivo imaging agent as defined in claim 1 wherein R¹ is methyl and R² is C₁₋₃ fluoroalkoxy.

4) The in vivo imaging agent as defined in claim 1 wherein R³ is hydrogen.

5) The in vivo imaging agent as defined in claim 1 wherein 2 of A¹, A² and A⁴⁻⁷ are N and the rest of A¹, A² and A⁴⁻⁷ are CH.

6) The in vivo imaging agent as defined in claim 1 wherein 1 of A² and A⁴-A⁶ is N; A¹ is CH; and, A⁷ is CH.

7) The in vivo imaging agent as defined in claim 1 wherein A⁷ is N; A¹⁻⁶ are CH; and, R³ is hydrogen.

8) The in vivo imaging agent as defined in claim 1 wherein said radioisotope is ¹⁸F, ¹¹C or ¹²³I.

9) The in vivo imaging agent as defined in claim 8 wherein said radioisotope is ¹⁸F.

10) The in vivo imaging agent as defined in claim 9 having the following chemical structure:

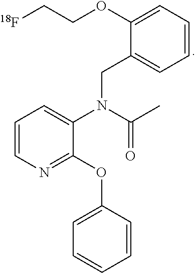

11) A method for the preparation of the in vivo imaging agent as defined in claim 1 comprising reaction of a suitable source of a radioisotope suitable for in vivo imaging with a precursor compound of Formula II: 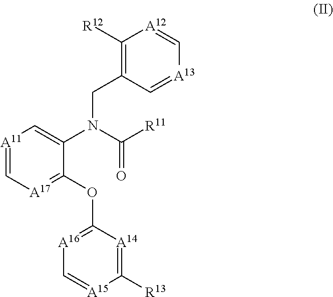
wherein:
one of R¹¹⁻¹³ comprises a precursor group; or
R¹¹ is methyl or C₁₋₃ fluoroalkyl, and optionally comprises a protecting group;
R¹² is hydrogen, halogen, C₁₋₃ alkoxy, or C₁₋₃ fluoroalkoxy, and optionally comprises a protecting group; and
R¹³ is hydrogen, halogen or C₁₋₃ alkoxy, and optionally comprises a protecting group;
A¹¹ is N or CH;
1 or 2 of A¹² and A¹⁴⁻¹⁷ are N, and the rest of A¹² and A¹⁴⁻¹⁷ are CH;
A¹³ is CH or CH—O—R⁴, where R⁴ is H, C₁₋₃ alkyl or C₁₋₃ haloalkyl, or A¹³ can alternatively be N when one of A¹² and A¹⁴⁻¹⁷ is N, and the rest of A¹² and A¹⁴⁻¹⁷ are CH;
and wherein A¹¹⁻¹⁷ optionally comprise a protecting group.

12) The method as defined in claim 11 wherein said suitable source of said radioisotope is a suitable source of ¹⁸F, and wherein said precursor group comprises hydroxyl, Cl, Br, I, tosylate, mesylate, or triflate.

13) The method as defined in claim 12 wherein said suitable source of ¹⁸F is [¹⁸F]-fluoroethyltosylate and said precursor compound is of the following chemical structure:

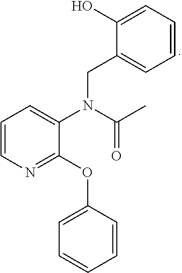

14) The method as defined in claim 12 wherein said suitable source of ¹⁸F is [¹⁸F]-fluoride and said precursor compound is of the following chemical structure: 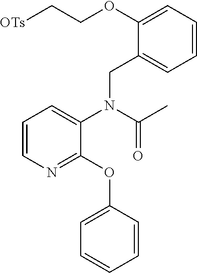
wherein OTs represents tosylate.

15) The method as defined in claim 11 wherein said suitable source of said radioisotope is a suitable source of ¹¹C, and wherein said precursor group is hydroxyl, carboxyl, a trialkylstannane, or an organoboron compound.

16) The method as defined in claim 11 wherein said suitable source of said radioisotope is a suitable source of ¹²³I, and wherein said precursor group is a trialkylstannane, a trialkylsilane, an organoboron compound, or, where said precursor group is attached to an aryl group of Formula II it forms, together with said aryl group, a phenol, an aryl iodonium salt, an aryl diazonium, an aryl trialkylammonium salt or a nitroaryl derivative.

17) The method as defined in claim 11 which is automated.

18) A precursor compound of Formula II: 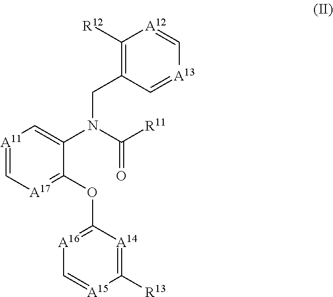
wherein:
one of R¹¹⁻¹³ comprises a precursor group; or
R¹¹ is methyl or C₁₋₃ fluoroalkyl, and optionally comprises a protecting group;
R¹² is hydrogen, halogen, C₁₋₃ alkoxy, or C₁₋₃ fluoroalkoxy, and optionally comprises a protecting group; and
R¹³ is hydrogen, halogen or C₁₋₃ alkoxy, and optionally comprises a protecting group;
A¹¹ is N or CH;
1 or 2 of A¹² and A¹⁴⁻¹⁷ are N, and the rest of A¹² and A¹⁴⁻¹⁷ are CH;
A¹³ is CH or CH—O—R⁴, where R⁴ is H, C₁₋₃ alkyl or C₁₋₃ haloalkyl, or A¹³ can alternatively be N when one of A¹² and A¹⁴⁻¹⁷ is N, and the rest of A¹² and A¹⁴⁻¹⁷ are CH;
and wherein A¹¹⁻¹⁷ optionally comprise a protecting group.

19) A radiopharmaceutical composition comprising the in vivo imaging agent as defined in claim 1 together with a biocompatible carrier in a form suitable for mammalian administration.

20) A kit for carrying out the method as defined in claim 11 wherein said kit comprises the precursor compound as defined in claim 18 in a sealed container.

21) A cassette for carrying out the method as defined in claim 17 comprising:
(i) a vessel containing the precursor compound as defined in claim 18; and,
(ii) means for eluting the vessel with a suitable source of radioisotope suitable for in vivo imaging.

22) The cassette as defined in claim 21 which additionally comprises:
(iii) an ion-exchange cartridge for removal of excess said radioisotope; and optionally,
(iv) a cartridge for deprotection of the resultant radiolabelled product to form an in vivo imaging agent as defined in claim 1.

23) An in vivo imaging method for use in determining the distribution and/or the extent of PBR expression in a subject comprising:
(i) administering to said subject an in vivo imaging agent as defined in claim 1;
(ii) allowing said in vivo imaging agent to bind to PBR in said subject;
(iii) detecting by an in vivo imaging procedure signals emitted by the radioisotope of said in vivo imaging agent;
(iv) generating an image representative of the location and/or amount of said signals; and,
(v) determining the distribution and extent of PBR expression in said subject wherein said expression is directly correlated with said signals emitted by said in vivo imaging agent.

24) The in vivo imaging method of claim 23 which is carried out repeatedly during the course of a treatment regimen for said subject, said regimen comprising administration of a drug to combat a PBR condition.

25) A method for diagnosis of a condition in which PBR is upregulated comprising the in vivo imaging method as defined in claim 23, together with the further step (vi) of attributing the distribution and extent of PBR expression to a particular clinical picture.

26) (canceled)

27) (canceled)
